(19) United States
(12) Patent Application Publication
(10) Pub. No.: US 2026/0257049 A1
(43) Pub. Date: Sep. 3, 2026

(54) INLINE PRESSURE RELIEF SYSTEM KIT FOR TRANSCUTANEOUS FILLING OF AN EMBEDDED DRUG RESERVOIR DELIVERY SYSTEM

(71) Applicant: CraniUS LLC, Baltimore, MD (US)

(72) Inventors: Bejan DARBANDI, Baltimore, MD (US); Owen FRIESEN, Baltimore, MD (US)

(73) Assignee: CraniUS LLC, Baltimore, MD (US)

(21) Appl. No.: 19/554,832

(22) Filed: Mar. 3, 2026

Related U.S. Application Data
(60) Provisional application No. 63/765,831, filed on Mar. 3, 2025.

(51) Int. Cl. A61M 39/02; A61M 31/00

(52) U.S. Cl. CPC A61M 39/0208; A61M 31/002; A61M 2205/3337; A61M 2209/045; A61M 2210/0687

(57) ABSTRACT

An inline pressure relief system kit for transcutaneous filling of an embedded drug reservoir is disclosed. The kit includes a pressure relief valve with a predetermined crack pressure, a refill syringe upstream of the valve, and a bypass syringe configured to receive fluid diverted when downstream pressure exceeds the crack pressure. A catheter and non-coring needle facilitate access to the reservoir. The system captures bypassed fluid to minimize drug waste and allow for re-injection. The kit may further include an inline filter to prevent the passage of cellular debris or bacterial emboli. A method for refilling a reservoir is also provided, comprising regulating fluid pressure during injection and diverting excess fluid to the bypass syringe to protect the reservoir from pressure-induced injury.

### CROSS-REFERENCE TO RELATED APPLICATIONS

This application claims the benefit of U.S. Provisional Application No. 63/765,831, filed on Mar. 3, 2025, the entire disclosure of which is hereby incorporated by reference.

### FIELD OF THE INVENTION

The present disclosure relates generally to implantable medical device refill systems and methods, and more particularly to an inline pressure relief system kit and method for the transcutaneous filling of an embedded drug reservoir featuring a bypass mechanism to regulate fluid pressure and minimize drug waste.

### BACKGROUND

The creation and safe use of refill systems is critical for implantable drug-delivery reservoir systems. Many such systems are at risk for overfilling and pressure-induced injuries to the internal reservoir, which can jeopardize both short-term and long-term device function, potentially leading to leakage and patient safety risks. Consequently, it is common practice for healthcare providers to be well-trained in instances involving transcutaneous needle fills to avoid these pressure-induced complications.

Aseptic (sterile) technique is absolutely crucial to the lifespan of an implanted drug reservoir. Because the skin serves as the primary barrier for bacterial protection, any needle penetrating the skin to access a “foreign body”—such as an implantable medicine delivery device—must be completely sterile. Failure to maintain a sterile fluid path and effective filling practices leads to a high risk of device infection and premature explantation, as the human body does not tolerate bacterial or fungal contamination within implanted devices.

A longstanding goal in the treatment of chronic disease is the development of successful delivery devices that reduce repetitive surgeries and ensure patient compliance. Such devices are particularly vital for treating brain diseases where pre-existing tissue barriers, such as the blood-brain barrier, render systemic delivery ineffective. For skull-embedded devices, repeatable and direct medicine delivery—such as chemotherapy or irradiation therapy—is necessary to match the growth stages of cancerous lesions. This requires a safe method of refilling the reservoir over time while precisely controlling and monitoring the volume and pressure of the medicine or imaging dye being introduced.

Existing refill kits often fail to provide adequate support in limiting unwanted air or cellular debris from entering the brain during transcutaneous injection. Furthermore, many current kits are designed with a small footprint. However, a short distance between the provider and the patient is not always desired. For example, when treating a brain tumor patient suffering from immunomodulation, a healthcare provider may prefer to maintain a distance to reduce the risk of nosocomial infection. Additionally, in the context of magnetic resonance imaging (MRI), short kits may be incompatible with equipment that limits the provider's proximity to the patient's head.

Many current kits also present detrimental risks related to clogging the internal fluid path. Blood particles, platelets, or brain particles can adhere to and obstruct the lumen of non-coring needles, tubing, or catheters, inhibiting effective device emptying. Reliably flushing current microcatheters is difficult, as existing microirrigators often fail to provide an effective male-to-female connection between the sterile saline syringe and the catheter. Finally, existing kits frequently utilize materials that are contraindicated for MRI environments, presenting a significant disadvantage to overall patient safety and diagnostic quality.

Additionally injecting fluid at too high a pressure in existing implanted pumps or reservoirs creates additional risk that the patient may be injured. However when utilizing existing refill kits there is no way for the administrator to properly measure the pressure relief. This also holds true for existing implantable pumps and reservoirs. To combat the risks of too high pressure existing protocols require that the drugs be infused over the course of a longer period of time, for example 5-10 minutes. This is painfully slow and often rushed which can result in patient feeling nausea, headache or other side effects.

### SUMMARY

According to at least one exemplary embodiment, an inline pressure relief system kit for transcutaneous filling of an embedded drug reservoir delivery system is disclosed. The kit provides a safety mechanism to prevent over-pressurization of implanted devices while maintaining a sterile fluid path and minimizing drug waste. The kit comprises a pressure relief valve configured to provide inline pressure regulation, one or more syringes connected upstream to the pressure relief valve, and one or more bypass devices connected to the pressure relief valve. In some embodiments, the kit may further comprise an attachment port connected to a first end of a syringe, and one or more stopcocks configured to control pressure or divert flow.

In another exemplary embodiment, the syringes include a refill syringe for delivering a fluid into the kit and a bypass syringe for receiving overflow fluid. The pressure relief valve is configured to divert fluid to the bypass syringe when a downstream pressure exceeds a predetermined crack pressure of the pressure relief valve. This configuration allows for the capture and subsequent re-injection of expensive or scarce medications, thereby reducing overall drug waste and improving healthcare cost-conscience efficiency.

According to another exemplary embodiment, the kit may include a non-coring needle for percutaneous access to a septum of an implanted reservoir, such as a skull-embedded medical device. An inline filter may be disposed within the fluid path to ensure the sterility and purity of the injection fluid by preventing cellular debris or bacterial emboli from moving into the patient's tissue. Furthermore, the catheter and associated tubing of the kit may be formed from biocompatible silicone or alloplastic materials, rendering the system MRI-safe and MRI-compatible.

According to another exemplary embodiment, a method for refilling an implanted drug reservoir is provided. The method includes connecting the kit components to an end device, depressing a main syringe to initiate fluid flow, and regulating pressure via an inline pressure relief valve. If fluid pressure exceeds the crack pressure of the valve, excess fluid is diverted to a bypass syringe, thereby protecting the reservoir from pressure-induced injury. The diverted fluid may then be returned to the main syringe to minimize waste.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 is an isometric view of an exemplary embodiment of an inline pressure relief system kit for transcutaneous filling of an embedded drug reservoir delivery system.

FIG. 2 is an isometric view of an exemplary embodiment of a pressure relief valve.

FIG. 3a is an isometric view of an exemplary embodiment of a syringe.

FIG. 3b is an isometric view of an exemplary embodiment of a stopcock.

FIG. 3c is an isometric view of an exemplary embodiment of a non-coring needle.

FIG. 3d is an isometric view of an exemplary embodiment of an inline filter.

FIG. 3e is an isometric view of an exemplary embodiment of a catheter.

FIG. 4 is a flow chart of an exemplary method to refill the kit utilizing a pressure sensitive reservoir.

### DETAILED DESCRIPTION

Aspects of the invention are disclosed in the following description and related drawings directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

As used herein MRI-safe may be understood to mean a device that when used in the MRI environment presents no additional risk to the patient or other individual, but it may affect the quality of the diagnostic information.

As used herein MRI-compatible may be understood to mean a device that is MRI safe when used in the MRI environment, and neither significantly affects the quality of the diagnostic information nor has its operations affected by the MRI system.

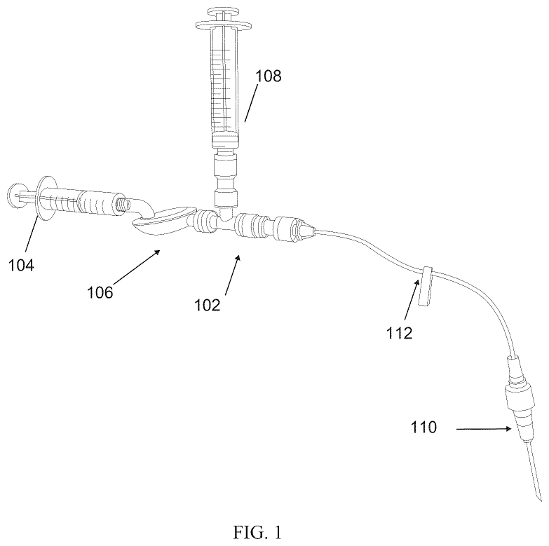

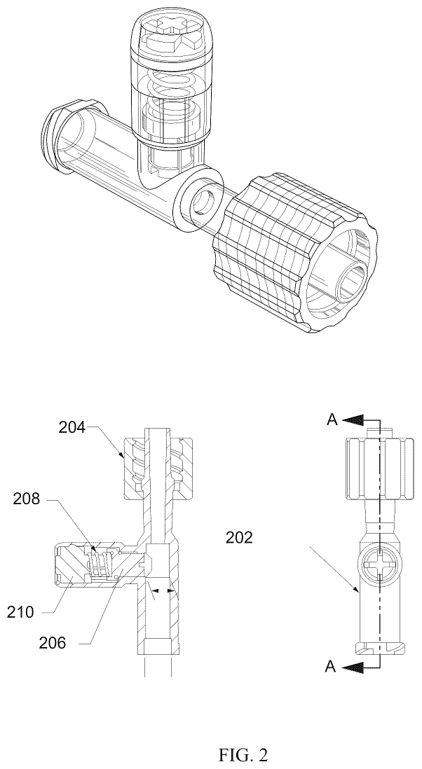

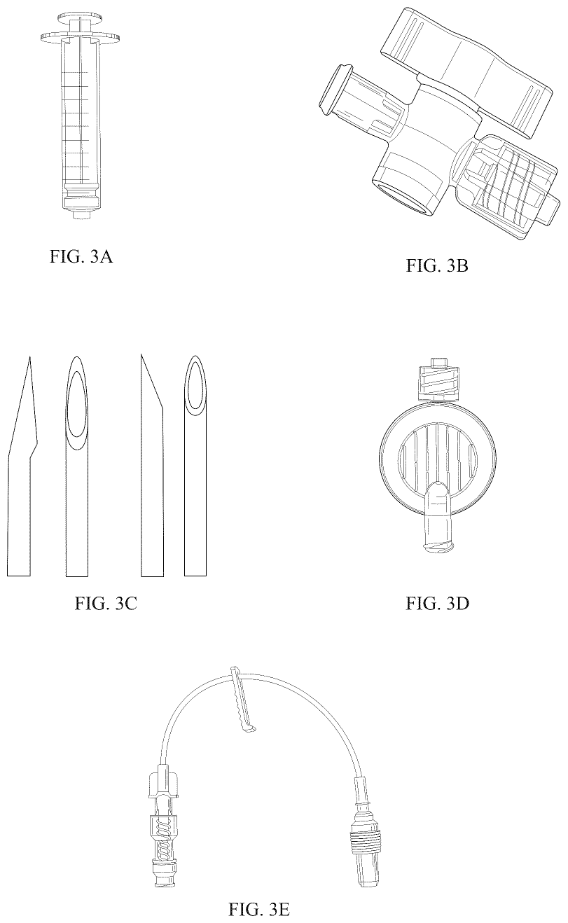

With reference to FIGS. 1-3, exemplary system architectures and related components may be shown and described. In one or more exemplary embodiments, an inline pressure relief system kit for transcutaneous filling of an embedded drug reservoir delivery system is provided. As shown in FIG. 1, the kit may include a pressure relief valve 102, illustrated in detail in FIG. 2, to provide inline pressure regulation. The kit further may include a first syringe 104 connected to the pressure relief valve via an in-line filter 106 and a bypass syringe 108 connected upstream of the pressure relief valve 102. The sizes of these syringes can be variable, and in some embodiments, the bypass side syringe 108 may consist of a syringe barrel only. The pressure relief valve 102 may be connected to a non-coring needle 110 via an IV tubing or catheter 112. As shown in FIG. 3a, the syringe body may be constructed of silicone or plastic. In some embodiments, the kit includes a refill syringe configured to inject or deliver a fluid (such as medication or saline) into the kit.

Exemplary systems for flow control and pressure regulation may be further illustrated in FIGS. 2 and 3b. As shown in FIG. 3b, the kit may optionally include one or more stopcocks to selectively control the pressure or divert flow. In some embodiments, the kit includes a bypass syringe featuring an interface such as a luer lock, slip tip, or other neuraxial connection configurations, for example as defined by ISO 80369-6. The pressure relief valve and the bypass syringe may be specifically configured to handle “overflow” when and if pressure limits are reached. The system captures excess medicine or imaging dye via the connected bypass syringe so that the fluid may be subsequently injected and not discarded. This minimizes drug waste, reduces healthcare costs, and preserves drug availability by reducing the risk of supply shortages. In some embodiments, an attachment port is connected to a first end of the syringe. In some embodiments the pressure relief valve 102 may include a connector body 202, a spin lock collar 204, a stem 206, a spring see table 208, and/or a plug 210.

Exemplary aspects of percutaneous access and sterility may be illustrated in FIGS. 3c and 3d. The kit may include a waste or bypass device connected to the pressure relief valve to ensure proper disposal or recovery of anything bypassed. This device may include a needle for percutaneous access to a septum or a reservoir. The needle is configured for attachment to a subcutaneous port entry within an implantable medical device, such as one embedded within a skull. As shown in FIG. 3c, the needle is preferably a non-coring needle. In some embodiments, the tube set and needle configurations may be variable to accommodate different clinical needs.

Exemplary filtration and patient safety features may be further illustrated in FIG. 3 d. The kit may optionally include an inline filter (e.g., a 0.2 μm filter) to ensure the sterility and purity of the injection fluid. The inline filter is configured so that flushed liquid enters the filter to prevent any risk of cellular debris or bacterial emboli moving into the brain during or after transcutaneous attachment. This maintenance of a sterile path is highly valuable in brain-related medicine delivery, where device obstruction or infection can have life-threatening consequences.

Exemplary catheter assembly and MRI environments may be illustrated in FIG. 3e. The kit includes a catheter. The catheter body is MRI-safe and MRI-compatible, formed from materials such as biocompatible silicone and/or an alloplastic material. The catheter may be configured to extend and lengthen the area between the refill syringe and the non-coring needle using additional intravenous (IV) tubing. This length allows for a larger distance footprint, which is critical when treating immunosuppressed patients to reduce nosocomial infection risks or when working with MRI equipment that limits proximity to the patient. The kit is specifically utilized in conjunction with a skull-embedded implant capable of achieving convection-enhanced delivery (CED). An additional key benefit of the IV extension tubing may be understood to serve as strain relief. This may allow a clinician to manipulate the syringe without moving the needle, which may minimize damage to the septum and the trauma of needle insertion.

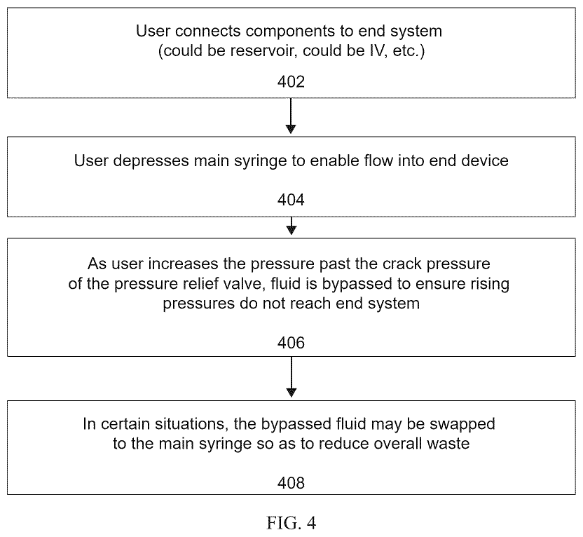

An exemplary method of operation for refilling a reservoir may be further described with reference to the flow chart of FIG. 4. In a first step 402, a user connects components (including the valve, syringes, and bypass devices) to an end device, such as a pressure-sensitive reservoir. In step 404, the user depresses a main syringe (the refill syringe or bypass syringe) to enable flow into the end device (the reservoir or catheter). In step 406, the kit facilitates bypassing of the fluid to ensure that rising pressures do not reach the end device as the user increases pressure past the crack pressure of the pressure relief valve. In step 408, the bypassed fluid is swapped back to the main syringe to reduce overall waste.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

1. An inline pressure relief system kit for transcutaneous filling of an embedded drug reservoir, comprising:
a pressure relief valve comprising a connector body, and a spring-biased stem, wherein the pressure relief valve has a predetermined crack pressure;
a refill syringe in fluid communication with and disposed upstream of the pressure relief valve;
a bypass syringe in fluid communication with the pressure relief valve, the bypass syringe configured to receive fluid diverted by the pressure relief valve when a downstream pressure of the valve exceeds the predetermined crack pressure, wherein the bypass syringe comprises a syringe barrel configured to capture and store diverted fluid in a sterile condition for subsequent re-injection into the refill syringe;
a catheter coupled to a downstream end of the pressure relief valve; and
a non-coring needle disposed at a distal end of the catheter for percutaneous access to the embedded drug reservoir.

2. The inline pressure relief system kit of claim 1, further comprising an inline filter disposed between the refill syringe and the non-coring needle, the filter having a pore size configured to prevent the passage of cellular debris or bacterial emboli.

3. The inline pressure relief system kit of claim 1, further comprising at least one stopcock disposed within a fluid path of the kit to selectively divert fluid flow or control pressure.

4. The inline pressure relief system kit of claim 1, wherein the bypass syringe has a luer lock or slip tip interface.

5. (canceled)

6. A method for refilling an implanted drug reservoir using an inline pressure relief kit comprising:
establishing fluid communication between a kit and an implanted reservoir by inserting a non-coring needle through a cutaneous layer;
depressing a plunger of a refill syringe to enable fluid to flow towards the implanted reservoir;
regulating fluid pressure via an inline pressure relief valve disposed between the refill syringe and the non-coring needle;
diverting a portion of the fluid into a bypass syringe when the fluid pressure exceeds a crack pressure of the pressure relief valve to prevent pressure-induced injury to the reservoir; and
recovering the diverted fluid from the bypass syringe while maintaining sterility of the diverted fluid for subsequent re-injection into the refill syringe, wherein the bypass syringe comprises a syringe barrel configured to capture and store the diverted fluid in a sterile condition.

7. The method of claim 6, wherein the implanted reservoir is a skull-embedded device configured for convection-enhanced delivery (CED) and/or intraventricular delivery.

8. The method of claim 6, further comprising filtering the fluid through an inline filter to prevent the cellular debris or bacterial emboli from entering the implanted reservoir;
wherein the inline filter has a pore size configured to prevent the passage of cellular debris or bacterial emboli.

9. The method of claim 6, further comprising selectively diverting the fluid flow or regulating pressure delivery to the implanted reservoir using at least one stopcock disposed within the kit.

10. The inline pressure relief system kit of claim 1, wherein the catheter is formed from MRI-safe and MRI-compatible materials comprising biocompatible silicone or alloplastic material.

11. The method of claim 6, wherein a catheter coupled between the pressure relief valve and the non-coring needle is formed from MRI-safe and MRI-compatible materials comprising biocompatible silicone or alloplastic material.
